(19) United States
(12) Patent Application Publication
(10) Pub. No.: US 2025/0268685 A1
(43) Pub. Date: Aug. 28, 2025

(54) METHOD FOR CARRYING OUT PATIENT REGISTRATION ON A MEDICAL VISUALIZATION SYSTEM, AND MEDICAL VISUALIZATION SYSTEM

(71) Applicant: CARL ZEISS MEDITEC AG, Jena (DE)

(72) Inventors: Johannes RANGEL, Aalen (DE); Stefan SAUR, Aalen (DE)

(21) Appl. No.: 19/058,224

(22) Filed: Feb. 20, 2025

(30) Foreign Application Priority Data
Feb. 22, 2024 (DE) 10 2024 201 661.6

(51) Int. Cl. A61B 90/00; G06T 7/00; G06T 7/33; G06T 7/73; G06T 17/20; G06T 19/20; G16H 10/60; G16H 40/20

(52) U.S. Cl. CPC A61B 90/361; G06T 7/0012; G06T 7/344; G06T 7/73; G06T 17/20; G06T 19/20; G16H 10/60; G16H 40/20; A61B 2090/367; G06T 2207/30004; G06T 2207/30204; G06T 2207/30244; G06T 2210/41; G06T 2219/2004; G06T 2219/2016

(57) ABSTRACT

A method and system include an image of a body part of a patient is captured by a camera of a medical visualization system, a trained machine learning method and/or a method of computer vision is used to estimate a three-dimensional surface profile of the body part in a coordinate system of the camera using the captured image as a starting point, a scaling factor of the three-dimensional surface profile is determined and/or estimated, and preoperatively acquired three-dimensional patient data that are available in a patient coordinate system are fitted to the estimated three-dimensional surface profile. A transformation rule between the coordinate system of the camera and the patient coordinate system is determined using a resultant fit result as a starting point, the fitting and/or the determining of the transformation rule is performed taking into account the determined and/or estimated scaling factor, and the determined transformation rule is provided.

The invention relates to a method for carrying out patient registration on a medical visualization system and to a medical visualization system.

In order to use preoperatively acquired data, such as for example computed tomography (CT) data or magnetic resonance imaging (MRI) data, in surgery, it is necessary to know a relationship between a reference coordinate system and a patient or patient coordinate system during the operation. Determining this relationship or a transformation rule, describing the relationship, between the coordinate systems is usually performed prior to the operation and is known as patient registration. The patient registration comprises in particular determining the relative pose (translation and rotation) of a patient in another coordinate system that does not move relative to the patient and therefore serves as reference coordinate system.

To register the patient, a surface of the patient and a pose of a reference object localized in the reference coordinate system may be determined, e.g. of a unique tracker or a unique marker that is affixed to the patient (for example a Mayfield clamp). The surface of the patient may be determined in the reference coordinate system e.g. by means of stereoscopic topography determination (for example derived from stereoscopic image data from a surgical microscope) or by means of scanning devices that operate in contact-based or contactless fashion, such as for example a Brainlab Softtouch or a Brainlab Z-Touch (both from Brainlab AG). The pose of the reference object (of the marker or tracker on the patient) may be captured and determined using a navigation system (for example from NDI, Canada) or, alternatively, using an internal tracking system of a medical visualization system.

EP 1 142 536 A1 has disclosed a method for referencing a patient or a body part of a patient in a camera-assisted, medical navigation system, including the following steps: the body part of the patient to be referenced is brought into the capture region of a navigation system assisted by at least two cameras, with the navigation system capturing the three-dimensional spatial positions of light markings with computer assistance, a light beam is used to create light markings on the surface of the body part to be referenced, with the three-dimensional position of said light markings being determined by the camera-assisted navigation system, the spatial pose of the surface of the body part to be referenced is determined using the position data for the light markings.

Further methods of patient registration are for example known from DE 10 2022 202 555 A1, DE 10 2022 100 626 A1 and US 2023/0 074 362 A1.

T. Mane et al., Single-camera 3D head fitting for mixed reality clinical applications, Computer Vision and Image Understanding, Vol. 218, April 2022, 103384, https://doi.org/10.1016/j.cviu.2022.103384, provides an overview of methods for estimating a three-dimensional surface profile.

D. Jiang et al., Efficient 3D reconstruction for face recognition Pattern Recognition, Vol. 38, No. 6, June 2005, pp. 787-798, https://doi.org/10.1016/j.patcog.2004.11.004, has disclosed methods of computer vision for estimating a three-dimensional surface profile.

Christian Wöhler, 3D Computer Vision-Efficient Methods and Applications, Springer Berlin, Heidelberg, 2009, https://doi.org/10.1007/978-3-642-01732-2, has disclosed methods of surface matching.

The invention is based on the problem of improving a method for carrying out patient registration on a medical visualization system and a medical visualization system.

According to the invention, the problem is solved by a method having the features of claim 1 and a medical visualization system having the features of claim 15. Advantageous configurations of the invention are evident from the dependent claims.

One of the basic concepts of the invention lies in estimating a three-dimensional surface profile of a body part of a patient by means of a trained machine method and/or a method of computer vision in a coordinate system of the camera, using an image captured by means of a camera of the medical visualization system as a starting point. For example, the camera is a main observer camera of the medical visualization system. Using this as a starting point, the estimated three-dimensional surface profile of the body part is fitted to preoperatively acquired three-dimensional patient data of the body part (e.g. computed tomography (CT) data or magnetic resonance imaging (MRI) data, etc.), for example by way of surface matching. The preoperatively acquired three-dimensional patient data being available in a patient coordinate system allows a fit result to determine a transformation rule between the coordinate system of the camera and the patient coordinate system. The preoperatively acquired three-dimensional patient data can subsequently be localized in the coordinate system of the camera by means of the determined transformation rule. For example, this allows the patient data captured preoperatively to be superimposed at the correct position onto images captured by means of the camera. A determined and/or estimated scaling factor of the three-dimensional surface profile is taken into account when adapting and/or determining the transformation rule.

In particular, a method for carrying out patient registration on a medical visualization system is made available, wherein an image of a body part of a patient is captured by means of a camera of the medical visualization system, wherein a trained machine learning method and/or a method of computer vision is used to estimate a three-dimensional surface profile of the body part in a coordinate system of the camera using the captured image as a starting point, wherein a scaling factor of the three-dimensional surface profile is determined and/or estimated, and wherein preoperatively acquired three-dimensional patient data that are available in a patient coordinate system are fitted to the estimated three-dimensional surface profile, wherein a transformation rule between the coordinate system of the camera and the patient coordinate system is determined using a resultant fit result as a starting point, wherein the fitting and/or the determining of the transformation rule is performed taking into account the determined and/or estimated scaling factor, and wherein the determined transformation rule is provided.

Further, a medical visualization system in particular is developed, comprising a camera configured to capture an image of a body part of a patient and a control device, wherein the control device is configured to estimate a three-dimensional surface profile of the body part in a coordinate system of the camera by means of a trained machine learning method and/or a method of computer vision, using the captured image as a starting point, to determine and/or estimate a scaling factor of the three-dimensional surface profile, to fit preoperatively acquired three-dimensional patient data that are available in a patient coordinate system to the estimated three-dimensional surface profile, to determine a transformation rule between the coordinate system of the camera and the patient coordinate system using a resultant fit result as a starting point, wherein the fitting and/or the determining of the transformation rule is performed taking into account the determined and/or estimated scaling factor, and to provide the determined transformation rule.

In particular, the method and the medical visualization system allow a patient registration to be performed with the aid of only a (single monoscopic) image. In particular, this allows effort, time and costs to be saved since it is possible in particular to manage without a stereoscopic capture and/or the successive point-by-point capture of the three-dimensional surface profile of the body part. In particular, the patient registration may be performed more expeditiously and in automated fashion as a result. In particular, the method and the medical visualization system render an automated marker-free patient registration possible. In particular, the method and the medical visualization system allow the provision of a robotic, geometrically calibrated visualization system.

In particular, the camera is a main observer camera of the medical visualization system. However, any other camera of the medical visualization system may also be used in principle. In particular, the camera is a monoscopic camera that captures monoscopic images. In particular, the camera is a (true) colour camera (RGB). However, in principle, the camera may also operate on the basis of other wavelengths (e.g. NIR).

The medical visualization system is in particular a surgical microscope. However, the medical visualization system may also be a microscope used for medical examinations and/or for diagnostic purposes. Furthermore, the medical visualization system may also be a handheld or hand-guided micro-inspection tool.

For example, the trained machine learning method may comprise a trained neural network or may be designed as such. In particular, the trained neural network is a deep neural network, in particular a convolutional neural network (CNN).

In particular, the machine learning method is trained to estimate a three-dimensional surface profile of the body part of the patient that is imaged in the captured image using said captured (in particular monoscopic) image as a starting point. For example, the three-dimensional surface profile may be estimated and/or described in the form of a three-dimensional mesh that consists of mesh points in particular. The machine learning method is or was trained with the aid of training data by way of supervised or unsupervised learning, especially within the scope of a training phase. The training data comprise pairs, in which a respective image of a body part is paired with a three-dimensional surface profile (as ground truth). In particular, this provides for a scaling factor of the three-dimensional surface structure to be taken into account such that the three-dimensional surface profile in coordinates (metric coordinates in particular) describes real dimensions of the body part. Alternatively, the three-dimensional surface profile may also be estimated in relative (arbitrarily chosen) coordinates. Such a surface profile may subsequently be scaled to the real dimensions by means of a suitable scaling factor. Then, the machine learning method is trained with the aid of the training data set, in particular by way of supervised learning. The training data may be created using captured images and captured surface profiles, in each case corresponding therewith, as a starting point. In an alternative to that or in addition, simulations may also be used to create pairs of images and associated surface profiles. For example, the machine learning method may be based on one of the methods described in T. Mane et al., Single-camera 3D head fitting for mixed reality clinical applications, Computer Vision and Image Understanding, Vol. 218, April 2022, 103384, https://doi.org/10.1016/j.cviu.2022.103384.

For example, a method of computer vision may be based on one of the methods described in D. Jiang et al., Efficient 3D reconstruction for face recognition Pattern Recognition, Vol. 38, No. 6, June 2005, pp. 787-798, https://doi.org/10.1016/j.patcog.2004.11.004. In particular, this provides for a database in which three-dimensional body models are stored to be used, in order to find the respective associated three-dimensional body model on the basis of two-dimensional features in the captured image.

In particular, the scaling factor allows relative distances in the three-dimensional surface profile to be assigned real distances or a real measure of length (e.g. in the metric system of units with the unit of “metre”). The scaling factor contains or describes such an assignment rule in particular.

Provision may be made for a camera pose of the camera to be determined in a reference coordinate system. In that case, the coordinate system of the camera corresponds to the reference coordinate system. An internal tracking system can be used to this end; for example, it comprises an environment camera of the medical visualization system and a marker (also referred to as tracker, for example a Mayfield clamp) arranged on the patient. A relative pose between the environment camera and the camera is known or may be determined by means of an extrinsic calibration. The environment camera is able to determine a pose of the marker arranged on the patient. Using this as a starting point, the camera pose may also be determined by way of the extrinsic calibration. Furthermore, it is possible to also use an external tracking system that captures both a marker arranged on the patient and a marker arranged on the camera and determines their respective poses. The camera pose may be determined using a known relative pose of the marker on the camera.

For example, the preoperatively acquired patient data are data acquired by means of a computed tomography (CT) scanner or by means of a magnetic resonance imaging (MRI) scanner. In principle, however, other preoperatively acquired patient data may also be used. In particular, the term preoperatively acquired patient data should also comprise three-dimensional patient data that were not acquired but for example simulated or created differently. The preoperatively acquired three-dimensional patient data are supplied to the control device and/or received by the latter.

For example, surface matching may be implemented by means of a method as described in Christian Wöhler, 3D Computer Vision-Efficient Methods and Applications, Springer Berlin, Heidelberg, 2009, https://doi.org/10.1007/978-3-642-01732-2.

Using the determined transformation rule, coordinates of the coordinate system of the camera or of a reference coordinate system are able to be converted into coordinates of the patient coordinate system and vice versa. In particular, using the transformation rule, the preoperatively acquired three-dimensional patient data are able to be localized in the coordinate system of the camera or in the reference coordinate system. In particular, this enables the patient data captured preoperatively to be superimposed at the correct position on a captured image from the camera for augmentation purposes. For example, the transformation rule may be expressed mathematically as follows:

$${P}_{m V} = {R}_{m V / Patient} * {P}_{P a t i ent} + {T}_{m V / Patient} ,$$

where P_{mV} has dimensions of 3×1 and denotes a point in the coordinate system of the camera (or in the reference coordinate system), P_{Patient} likewise has dimensions of 3×1 and denotes a point in the patient coordinate system, R_{mV/Patient} is a matrix that has dimensions of 3×3 and as a linear map describes a rotation and T_{mV/Patient} denotes a translation vector which has dimensions of 3×1.

Parts of the medical visualization system, in particular the control device, may be designed, either individually or together, as a combination of hardware and software, for example as program code that is executed on a microcontroller or microprocessor. However, provision may also be made for parts to be designed, either individually or together, as an application-specific integrated circuit (ASIC) and/or a field-programmable gate array (FPGA). In particular, the control device comprises at least one computing device and at least one memory.

The method and the medical visualization system may be used in particular in the following fields: neurosurgery, spinal surgery, dental surgery, eye surgery, etc.

One embodiment provides for the scaling factor of the estimated three-dimensional surface profile to be determined using settings of the camera and/or of optical elements of the medical visualization system at the time of image capture as a starting point. The settings of the camera and/or of the optical elements relate in particular to operating parameters when the image is captured, in particular a working distance and/or a focus and/or a magnification. The operating parameters may be used to determine a distance between points in the captured image and to determine the scaling factor thereby.

One developing embodiment provides for the scaling factor to be determined as follows: retrieving and/or ascertaining at least one focal value and one magnification value as settings of the medical visualization system; determining a ratio between a relative distance between selected points of the three-dimensional surface profile and an absolute distance between the selected points, the latter distance being determined from a known picture element size of an image sensor of the camera, with at least the focal value and the magnification value being taken into account; providing the determined ratio as scaling factor. A physical size (e.g. in metres or millimetres) corresponding to a picture element in the captured image is determined by a focal value (or working distance), a magnification value and a physical size of a picture element of an image sensor of the camera. If consideration is given to two three-dimensional points from the estimated surface profile which correspond to picture elements in the captured image, and if the two points are additionally assumed, to a first approximation, to be located in a focal plane corresponding to the focal value, then it is possible to determine an absolute distance between the three-dimensional points. Using this distance as a starting point, it is possible to determine the scaling factor for the three-dimensional surface profile as the ratio between the relative and absolute distances of the points. Subsequently, it is possible to provide the three-dimensional surface profile, in particular with an absolute measure of length (e.g. in units of metres).

One embodiment provides for the scaling factor of the estimated three-dimensional surface profile to be determined using as a starting point at least one marker that is arranged on the patient and that is captured by means of the camera or by the environment camera. As a result, the scaling factor may be determined using known properties (in particular known dimensions) of the at least one marker, which has a known 3-D geometry in particular, as a starting point.

One developing embodiment provides for the following steps to be carried out to this end: identifying the at least one marker in the image captured by means of the camera; determining properties of the identified at least one marker using the captured image as a starting point; determining a ratio between the determined properties and known properties of the identified at least one marker; providing the determined ratio as scaling factor. The scaling factor for the three-dimensional surface profile may be determined using the known properties (in particular known dimensions) as a starting point. Since the picture elements of the image correspond directly to the estimated three-dimensional surface profile, this can be used as a starting point to infer properties (in particular dimensions) of the three-dimensional surface profile on the basis of the known properties (in particular the known dimensions) of the marker, and so the scaling factor can be determined by way of the ratio. For example, a relative distance of the marker in the image may be related to a (known) absolute distance of the marker. In a simple case, the marker may comprise a length scale arranged on the patient, from which a measure of length may be determined directly.

Another developing embodiment provides for the following steps to be carried out to this end: identifying the at least one marker in an environment image captured by means of the environment camera; projecting the identified at least one marker into the captured image with a known relative pose between the environment camera and the camera being taken into account; determining properties of the projected at least one marker using the captured image as a starting point; determining a ratio between the determined properties and known properties of the projected at least one marker; providing the determined ratio as scaling factor. Since the relative pose between the camera (or the camera coordinate system) and a coordinate system of the environment camera is known (or able to be determined by means of an extrinsic calibration), a marker identified by means of the environment camera may be projected true-to-scale into the image of the camera. Using this and known properties of the marker (in particular its dimensions) as a starting point, it is possible to determine the scaling factor of the estimated three-dimensional surface profile since the picture elements in the image correspond to the estimated three-dimensional surface profile. In other words, the required metric information originates in particular from the marker (i.e., in particular, a known 3-D geometry of the marker and an estimated 6-D pose in relation to the coordinate system of the camera). The 3-D positions of the marker are projected on the image of the camera, in particular with the aid of the intrinsic calibration parameters of the camera, i.e. the 2-D position of the marker is determined in the image in particular. This creates a “mapping” between 3-D and 2-D marker positions, by means of which it is possible to calculate the scaling factor.

One embodiment provides for a camera pose of the camera to be determined in a reference coordinate system, wherein the transformation rule is determined between the reference coordinate system and the patient coordinate system. As a result, it is possible to refer to the reference coordinate system directly.

One embodiment provides for a change in a camera pose of the camera to be followed by a renewed capture of an image of the body part of the patient and renewed fitting and the renewed determination of the transformation rule. As a result, the transformation rule can be determined anew for each new camera pose. This can improve an accuracy of the transformation rule following a change in the camera pose.

One embodiment provides for the trained machine learning method and/or the method of computer vision to comprise at least one first method and at least one second method, wherein the at least one first method determines distinguished points on the body part in the captured image, and wherein the at least one second method estimates the three-dimensional surface profile using the determined distinguished points as a starting point. In other words, the step in which the three-dimensional surface profile of the body part is estimated in a coordinate system of the camera may be implemented in particular by virtue of distinguished points on the body part being determined in the captured image and the three-dimensional surface profile being estimated using the determined distinguished points as a starting point. This may realize an incremental evaluation of the captured image. In particular, this may increase an accuracy since the individual methods may each be specialized for the partial tasks only. In particular, distinguished points are regions that are as rigid and/or bony as possible, and/or predefined or predetermined regions. In other words, distinguished points in particular do not coincide with regions on the body part which may change (significantly) on account of a mobility/flexibility of the outer tissue (e.g. skin). For example, in the case of craniotomy, prominent points in the region of the nasal bone, frontal bone and/or cheekbone may be used as distinguished points. For example, the three-dimensional surface profile may be estimated by means of the at least one second method by virtue of the determined distinguished points being assigned to a three-dimensional model or a plurality of three-dimensional models of the body part (which may also be referred to as “mapping”) and being deformed in three dimensions. Provision may be made for the at least one second method to be provided with a three-dimensional model of the respective body part and/or for such a model to be used by the at least one second method for the purpose of estimating the three-dimensional surface profile.

One embodiment provides for the camera to be an environment camera of the medical visualization system. This allows a larger capture region to be captured than with a main observer camera for example. In particular, the larger capture region enables simultaneous capture of a marker arranged on the patient (e.g. Mayfield clamp).

One embodiment provides for at least one further image of the body part of the patient to be captured in at least one other camera pose of the camera or by means of a further camera of the medical visualization system arranged in the at least one other camera pose, wherein fitting is implemented with the captured at least one further image being taken into account. This may improve an accuracy when determining the transformation rule. For example, the camera pose and the at least one other camera pose of the camera may be set by means of a robotic stand of the medical visualization system.

One embodiment provides for a three-dimensional surface profile of the body part to be likewise estimated by means of the trained machine learning method and/or the method of computer vision using the captured at least one further image as a starting point, wherein the three-dimensional surface profile of the body part estimated from the at least one further image is fused with the three-dimensional surface profile of the body part estimated from the captured image, wherein fitting is implemented using the fused three-dimensional surface profile as a starting point. This allows a three-dimensional surface profile to be estimated separately for each of the captured images; the results, i.e. the respective individually estimated three-dimensional surface profiles, are only subsequently fused with one another. By fusing the estimated three-dimensional surface profiles, it is possible in particular to achieve a more accurate estimate of the three-dimensional geometry (or of the surface profile).

One embodiment provides for a three-dimensional surface profile of the body part to be likewise estimated by means of the trained machine learning method and/or the method of computer vision using the captured at least one further image as a starting point, wherein fitting to the preoperatively acquired three-dimensional patient data is additionally also implemented for the three-dimensional surface profile estimated using the captured at least one further image as a starting point, wherein the transformation rule is determined taking into account the fit results. As a result, the transformation rule can be determined using fits performed individually in each case as a starting point.

One embodiment provides for the camera and/or the at least one further camera for capturing the images to be arranged in at least two camera poses by means of a robotic stand of the medical visualization system. As a result, images can be set and captured in the plurality of camera poses, in particular in automated fashion. The automated capture allows the images to be captured systematically and in reproducible fashion. Errors resulting from manually setting a camera pose can be avoided.

The invention is explained in greater detail below on the basis of preferred exemplary embodiments with reference to the figures. In the figures:

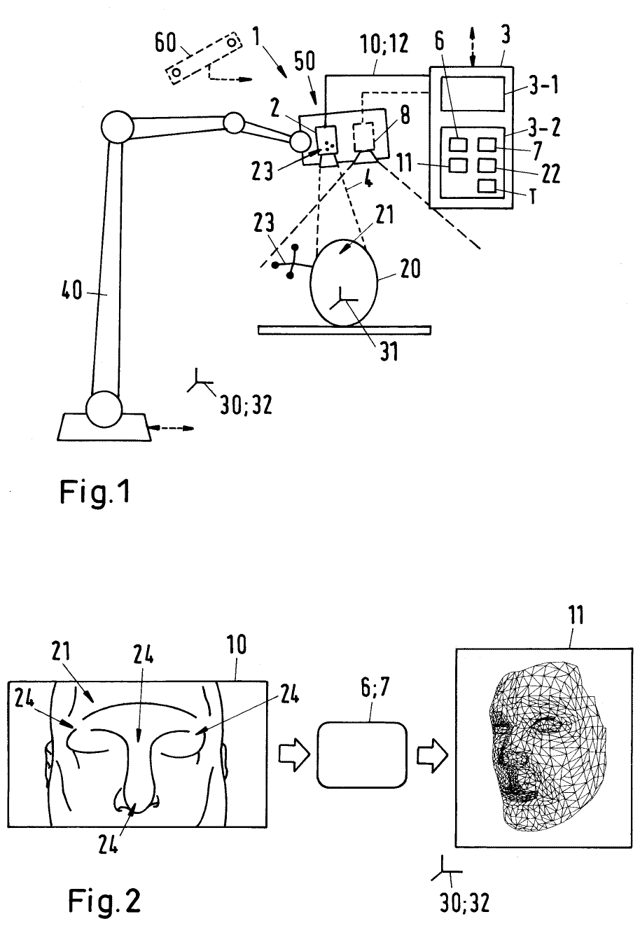

FIG. 1 shows a schematic illustration for elucidating embodiments of the medical visualization system and of the method;

FIG. 2 shows a schematic illustration for elucidating the estimation of the three-dimensional surface profile;

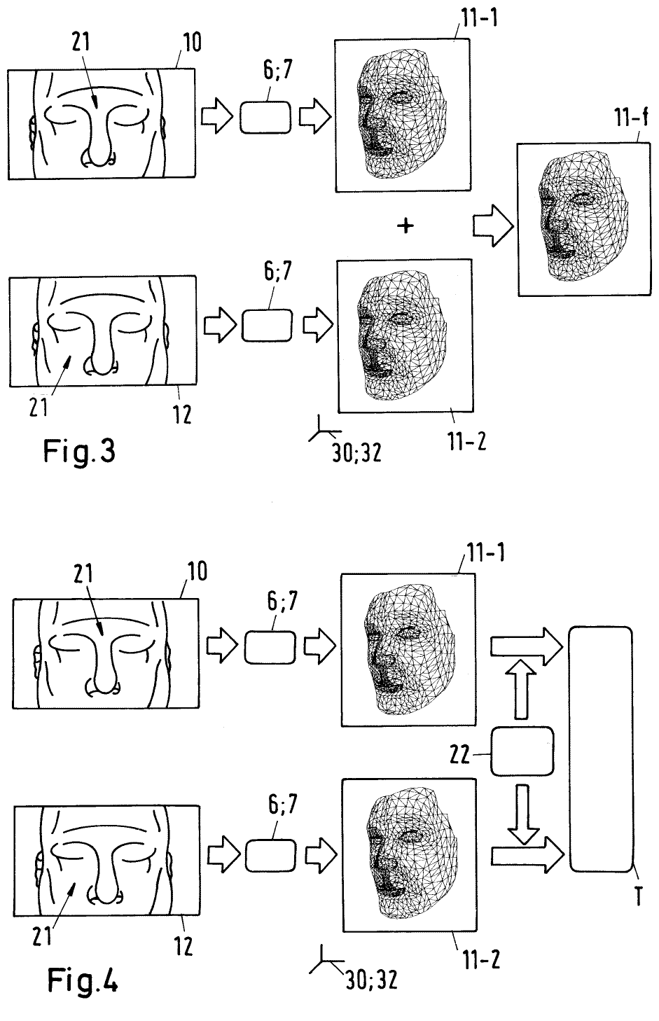

FIG. 3 shows a schematic illustration for elucidating an embodiment of the medical visualization system and of the method;

FIG. 4 shows a schematic illustration for elucidating an embodiment of the medical visualization system and of the method;

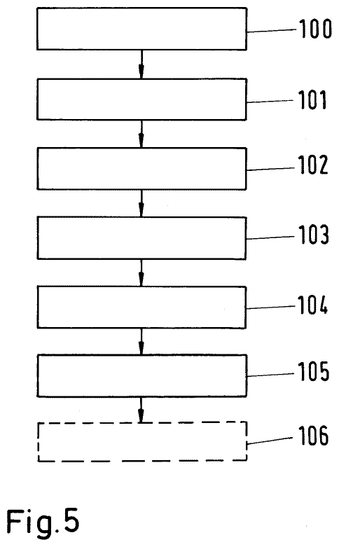

FIG. 5 shows a schematic flowchart for elucidating embodiments of the method.

FIG. 1 shows a schematic illustration for elucidating embodiments of the medical visualization system 1. The medical visualization system 1 comprises a camera 2 (in particular a single, more particularly monoscopic camera) and a control device 3. Further, the medical visualization system 1 may comprise a robotic stand 40, at the distal end of which the camera 2 is arranged. For example, the robotic stand 40 is controlled by the control device 3. The method described in this disclosure is explained in detail below on the basis of the medical visualization system 1.

The camera 2 is configured to capture an image 10 of a body part 21 of a patient 20. To this end, the body part 21, for example the head of the patient 20, is arranged in a capture region 4 of the camera 2 and captured. To this end, the camera 2 is arranged in a camera pose 50, for example by means of the robotic stand 40. The camera 2 captures an image 10 of the body part 21 and supplies said image to the control device 3.

The control device 3 comprises at least one computing device 3-1 and at least one memory 3-2. The control device 3 is configured to estimate a three-dimensional surface profile 11 (cf. also FIG. 2) of the body part 21 in a coordinate system 30 of the camera 2 by means of a trained machine learning method 6 and/or a method of computer vision 7, using the captured image 10 as a starting point. To this end, the trained machine learning method 6 is provided by the control device 3. In particular, a structure description and parameters of the trained machine learning method 6 are stored in the memory 5 to this end. In an alternative to that or in addition, the method of computer vision 7 is provided by the control device 3, wherein a description of the method is stored in the memory 3-2.

The control device 3 is also configured to determine and/or estimate a scaling factor of the three-dimensional surface profile 11.

The control device 3 is also configured to fit preoperatively acquired three-dimensional patient data 22 that are available in a patient coordinate system 31 to the estimated three-dimensional surface profile 11. For example, this is implemented by way of surface matching.

The control device 3 is furthermore configured to determine a transformation rule T between the coordinate system 30 of the camera 2 and the patient coordinate system 31 using a resultant fit result as a starting point.

Provision is made for the fitting and/or the determining of the transformation rule T to be performed taking into account the determined and/or estimated scaling factor.

The control device 3 is configured to provide the determined transformation rule T. In particular, the provision may comprise loading the determined transformation rule T into the memory 3-2 such that the transformation rule T can subsequently be used, in particular in order to superimpose the preoperatively acquired patient data 22 on a captured image 10 of the body part 21 for augmentation purposes.

FIG. 2 shows schematic illustrations for elucidating the estimation of the three-dimensional surface profile 11. In this case, the three-dimensional surface profile 11 is estimated in the coordinate system 30 of the camera. The three-dimensional surface profile 11 is subsequently fitted to the preoperatively acquired three-dimensional patient data, and the transformation rule is determined using the resultant fit result as a starting point. The fitting and/or the determining of the transformation rule T is performed taking into account the determined and/or estimated scaling factor.

Provision may be made for the scaling factor of the estimated three-dimensional surface profile 11 to be determined using settings of the camera 2 and/or of optical elements of the medical visualization system 1 at the time of image capture as a starting point. In particular, the settings may comprise operating parameters, for example a working distance and/or a focus and/or a magnification. In particular, the settings comprise a focus and a magnification. Using this as a starting point, it is possible to determine metric coordinates of the three-dimensional surface profile 11.

To this end, provision may be made for the scaling factor to be determined as follows: retrieving and/or ascertaining at least one focal value and one magnification value as settings of the medical visualization system 1; determining a ratio between a relative distance between selected points of the three-dimensional surface profile 11 and an absolute distance between the selected points, the latter distance being determined from a known picture element size of an image sensor of the camera 2, with at least the focal value and the magnification value being taken into account; providing the determined ratio as scaling factor.

Provision may be made for the scaling factor of the estimated three-dimensional surface profile 11 to be determined using as a starting point at least one marker 23 that is arranged on the patient 20 and that is captured by means of the camera 2 or by the environment camera 8 (FIG. 1) of the medical visualization system 1 and to be used when determining the transformation rule T. In particular, a pose of the camera 2 relative to the marker 23 may be determined by means of the environment camera 8. In particular, this requires an extrinsic calibration, i.e. the determination of a relative pose between the environment camera 8 and the camera 2. In that case, the coordinate system 30 of the camera 2 is defined by way of the pose of the marker 23 in particular.

Provision may be made for the following steps to be carried out to this end: identifying the at least one marker 23 in the image 10 captured by means of the camera 2; determining properties of the identified at least one marker 23 using the captured image 10 as a starting point; determining a ratio between the determined properties and known properties of the identified at least one marker 23; providing the determined ratio as scaling factor. In particular, the properties relate at least to measurements in at least one dimension of the at least one marker 23. For example, the ratio may be determined between a relative distance of the marker 23 in the image and a (known) absolute distance of the marker 23.

Provision may be made for the following steps to be carried out to this end: identifying the at least one marker 23 in an environment image captured by means of the environment camera 8; projecting the identified at least one marker 23 into the captured image 10 with a known relative pose between the environment camera 8 and the camera 2 being taken into account; determining properties of the projected at least one marker 23 using the captured image 10 as a starting point; determining a ratio between the determined properties and known properties of the projected at least one marker 23; providing the determined ratio as scaling factor. In particular, the properties relate at least to measurements in at least one dimension of the at least one marker 23.

Provision may be made for a camera pose 50 of the camera 2 to be determined in a reference coordinate system 32, wherein the transformation rule T is determined between the reference coordinate system 32 and the patient coordinate system 31. This may be implemented by means of an internal tracking system, for example by means of an environment camera 8 and a marker 23 arranged on the patient 20, as already described above. In an alternative to that or in addition, the camera pose 50 may also be determined by means of an external tracking system 60 (e.g. from NDI, Canada) and a marker 23 arranged on the camera 2. The three-dimensional surface profile 11 is then estimated in the reference coordinate system 32. In particular, provision is made for the determined camera pose 50 to be supplied to the trained machine learning method 6 and/or the method of computer vision 7 as input data such that said methods are able to determine the three-dimensional surface profile 11 with respect to the reference coordinate system 32.

Provision may be made for a change in the camera pose 50 of the camera 2 to be followed by a renewed capture of an image 10 of the body part 21 of the patient 20 and renewed fitting and the renewed determination of the transformation rule T. In particular, this may be implemented in the event of any change in the camera pose 50.

Provision may be made for the trained machine learning method 6 and/or the method of computer vision 7 to comprise at least one first method and at least one second method, wherein the at least one first method determines distinguished points 24 (FIG. 2) on the body part 21 in the captured image 10, and wherein the at least one second method estimates the three-dimensional surface profile 11 using the determined distinguished points 24 as a starting point. Some distinguished points 24, for example a tip of the nose, a root of the nose and the eyebrows, are elucidated in exemplary and schematic fashion in FIG. 2. The determined distinguished points 24 are transferred to the at least one second method, and using this as a starting point, the at least one second method estimates the three-dimensional surface profile 11, in particular with a scaling being taken into account.

Provision may be made for the camera 2 to be an environment camera 8 of the medical visualization system 1. In other words, the method may also be performed by means of the environment camera 8. The procedure is basically analogous here. In particular, the environment camera 8 is also a monoscopic camera that captures monoscopic images.

Provision may be made for at least one further image 12 of the body part 21 of the patient 20 to be captured in at least one other camera pose 50 of the camera 2 or by means of a further camera (not shown here) of the medical visualization system 1 arranged in the at least one other camera pose 50, wherein fitting is implemented with the captured at least one further image 12 being taken into account.

Provision may be made for a three-dimensional surface profile 11-2 of the body part 21 to be likewise estimated by means of the trained machine learning method 6 and/or the method of computer vision 7 using the captured at least one further image 12 as a starting point, wherein the three-dimensional surface profile 11-2 of the body part 21 estimated from the at least one further image 12 is fused with the three-dimensional surface profile 11-1 of the body part 21 estimated from the captured image 10, wherein fitting is implemented using the fused three-dimensional surface profile 11-f as a starting point. This embodiment is elucidated schematically in FIG. 3.

Provision may be made for a three-dimensional surface profile 11-2 of the body part 21 to be likewise estimated by means of the trained machine learning method 6 and/or the method of computer vision 7 using the captured at least one further image 12 as a starting point, wherein fitting to the preoperatively acquired three-dimensional patient data 22 is additionally also implemented for the three-dimensional surface profile 11-2 estimated using the captured at least one further image 12 as a starting point, wherein the transformation rule T is determined taking into account the fit results. In particular, all available fit results are taken into account when determining the transformation rule T. This embodiment is elucidated schematically in FIG. 4.

Provision may also be made for the camera 2 and/or the at least one further camera for capturing the images 10, 12 to be arranged in at least two camera poses 50 by means of the robotic stand 40 (FIG. 1) of the medical visualization system 1.

FIG. 5 shows a schematic flowchart for elucidating embodiments of the method for carrying out patient registration on a medical visualization system. For example, the method may be performed by means of a medical visualization system as described with reference to FIG. 1.

In a method step 100, an image of a body part of a patient is captured by means of a camera (in particular a monoscopic camera) of the medical visualization system. In this case, a camera pose while the image is captured is known in a coordinate system of the camera.

In a method step 101, a trained machine learning method and/or a method of computer vision is used to estimate a three-dimensional surface profile of the body part in a coordinate system of the camera using the captured image as a starting point. In this case, the three-dimensional surface profile is estimated with respect to the coordinate system of the camera in particular; i.e., coordinates of points of the three-dimensional surface profile are available in the coordinate system of the camera following the estimation. As already described above, the coordinate system of the camera may also be a reference coordinate system.

In a method step 102, a scaling factor of the three-dimensional surface profile is determined and/or estimated. In principle, method step 102 may also be part of method step 101.

In a method step 103, preoperatively acquired three-dimensional patient data that are available in a patient coordinate system are fitted to the estimated three-dimensional surface profile. In particular, this is implemented by way of surface matching.

In a method step 104, a transformation rule is determined between the coordinate system of the camera and the patient coordinate system using a resultant fit result as a starting point. In particular, the transformation rule contains a coordinate transformation between the coordinate system of the camera (or the reference coordinate system) and the patient coordinate system. In particular, parameters of this coordinate transformation are determined within the scope of the determination.

The transformation rule (T) is fitted in method step 103 and/or determined in method step 104 taking into account the scaling factor determined and/or estimated in method step 102.

The determined transformation rule is provided in a method step 105. In particular, the provision may comprise loading the transformation rule into a memory of the medical visualization system such that the transformation rule can subsequently be used.

In an optional method step 106, provision may be made for the determined transformation rule to be used to superimpose on the pre-operatively acquired patient data a captured image in order to provide an augmented image thereby. As a result, a user of the medical visualization system, in particular a surgeon, may be supported during a procedure and/or during an examination.

Further embodiments of the method have already been described with reference to the medical visualization system.

### LIST OF REFERENCE SIGNS

1 Medical visualization system
2 Camera
3 Control device
3-1 Computing device
3-2 Memory
4 Capture region
6 Trained machine learning method
7 Method of computer vision
8 Environment camera
10 Captured image
11 Three-dimensional surface profile
11-1 Three-dimensional surface profile
11-2 Three-dimensional surface profile
11-f Fused three-dimensional surface profile
12 Further image
20 Patient
21 Body part
22 Preoperatively acquired three-dimensional patient data
23 Marker
24 Distinguished point
30 Coordinate system (camera)
31 Patient coordinate system
32 Reference coordinate system
40 Robotic stand
50 Camera pose
60 External tracking system
100-106 Method steps
T Transformation rule

1. A method for carrying out patient registration on a medical visualization system, comprising:
capturing an image of a body part of a patient by a camera of the medical visualization system,
estimating, with a trained machine learning method and/or a method of computer vision, a three-dimensional surface profile of the body part in a coordinate system of the camera using the captured image as a starting point,
determining or estimating a scaling factor of the three-dimensional surface profile,
fitting preoperatively acquired three-dimensional patient data that are available in a patient coordinate system to the estimated three-dimensional surface profile,
determining a transformation rule between the coordinate system of the camera and the patient coordinate system using a resultant fit result as a starting point,
wherein the determining of the transformation rule is performed taking into account the determined and/or estimated scaling factor, and
providing the determined transformation rule.

2. The method according to claim 1, wherein the scaling factor of the estimated three-dimensional surface profile is determined using settings of the camera and/or of optical elements of the medical visualization system at the time of image capture as a starting point.

3. The method according to claim 2, wherein the scaling factor is determined as follows:
retrieving and/or ascertaining at least one focal value and one magnification value as settings of the medical visualization system;
determining a ratio between a relative distance between selected points of the three-dimensional surface profile and an absolute distance between the selected points, the latter distance being determined from a known picture element size of an image sensor of the camera, with at least the focal value and the magnification value being taken into account;
providing the determined ratio as scaling factor.

4. The method according to claim 1, wherein the scaling factor of the estimated three-dimensional surface profile is determined using as a starting point at least one marker that is arranged on the patient and that is captured by means of the camera or by the environment camera of the medical visualization system.

5. The method according to claim 4, further comprising:
identifying the at least one marker in the image captured by means of the camera;
determining properties of the identified at least one marker using the captured image as a starting point;
determining a ratio between the determined properties and known properties of the identified at least one marker; and
providing the determined ratio as scaling factor.

6. The method according to claim 4, further comprising:
identifying the at least one marker in an environment image captured by means of the environment camera;
projecting the identified at least one marker into the captured image with a known relative pose between the environment camera and the camera being taken into account;
determining properties of the projected at least one marker using the captured image as a starting point;
determining a ratio between the determined properties and known properties of the projected at least one marker; and
providing the determined ratio as scaling factor.

7. The method according to claim 1, wherein a camera pose of the camera is determined in a reference coordinate system, wherein the transformation rule is determined between the reference coordinate system and the patient coordinate system.

8. The method according to claim 1, wherein a change in a camera pose of the camera is followed by a renewed capture of an image of the body part of the patient and renewed fitting and the renewed determination of the transformation rule.

9. The method according to claim 1, wherein the trained machine learning method and/or the method of computer vision comprises at least one first method and at least one second method, wherein the at least one first method determines distinguished points on the body part in the captured image, and wherein the at least one second method estimates the three-dimensional surface profile using the determined distinguished points as a starting point.

10. The method according to claim 1, wherein the camera is an environment camera of the medical visualization system.

11. The method according to claim 1, wherein at least one further image of the body part of the patient is captured in at least one other camera pose of the camera or by means of a further camera of the medical visualization system arranged in the at least one other camera pose, wherein fitting is implemented with the captured at least one further image being taken into account.

12. The method according to claim 11, wherein a three-dimensional surface profile of the body part is likewise estimated by means of the trained machine learning method and/or the method of computer vision using the captured at least one further image as a starting point, wherein the three-dimensional surface profile of the body part estimated from the at least one further image is fused with the three-dimensional surface profile of the body part estimated from the captured image, wherein fitting is implemented using the fused three-dimensional surface profile as a starting point.

13. The method according to claim 11, wherein a three-dimensional surface profile of the body part is likewise estimated by means of the trained machine learning method and/or the method of computer vision using the captured at least one further image as a starting point, wherein fitting to the preoperatively acquired three-dimensional patient data is additionally also implemented for the three-dimensional surface profile estimated using the captured at least one further image as a starting point, wherein the transformation rule is determined taking into account the fit results.

14. The method according to claim 11, wherein the camera and/or the at least one further camera for capturing the images is arranged in at least two camera poses by means of a robotic stand of the medical visualization system.

15. A medical visualization system, comprising:
a camera configured to capture an image of a body part of a patient and a control device,
wherein the control device is configured
to estimate a three-dimensional surface profile of the body part in a coordinate system of the camera by means of a trained machine learning method and/or a method of computer vision, using the captured image as a starting point,
to determine and/or estimate a scaling factor of the three-dimensional surface profile,
to fit preoperatively acquired three-dimensional patient data that are available in a patient coordinate system to the estimated three-dimensional surface profile,
to determine a transformation rule between the coordinate system of the camera and the patient coordinate system using a resultant fit result as a starting point,
wherein the fitting and/or the determining of the transformation rule is performed taking into account the determined and/or estimated scaling factor, and
to provide the determined transformation rule.
